(19) United States
(12) Patent Application Publication
(10) Pub. No.: US 2023/0172941 A1
(43) Pub. Date: Jun. 8, 2023

(54) PROPHYLAXIS AND TREATMENT OF PATHOGENIC CORONAVIRUS INFECTIONS

(71) Applicant: The Johns Hopkins University, Baltimore, MD (US)

(72) Inventor: Ephraim Fuchs, Baltimore, MD (US)

(21) Appl. No.: 17/911,620

(22) PCT Filed: Mar. 18, 2021
(86) PCT No.: PCT/US2021/023033

§ 371 Date: Sep. 14, 2022

Related U.S. Application Data
(60) Provisional application No. 62/992,243, filed on Mar. 20, 2020.

(51) Int. Cl. A61K 31/52; A61K 31/496; A61K 31/416; A61P 31/14

(52) U.S. Cl. CPC A61K 31/52; A61K 31/416; A61K 31/496; A61P 31/14

(57) ABSTRACT

Methods for preventing or ameliorating decreased pulmonary function and pulmonary inflammation in a subject infected with SARS coronavirus such as SARS-CoV-2 by administration of certain tyrosine kinase inhibitors such as ibrutinib to the subject are provided.

### CROSS-REFERENCE TO RELATED APPLICATIONS

This application claims the benefit under 35 USC § 119(e) to U.S. Application Serial No. 62/992,243 filed Mar. 20, 2020, now pending. The disclosure of the prior application is considered part of and is incorporated by reference in the disclosure of this application.

### FIELD OF THE INVENTION

The present invention relates generally to immune responses and more specifically to methods for altering the T-cell response in a subject infected with a pathogen.

### BACKGROUND OF THE INVENTION

On Mar. 11, 2020, the World Health Organization declared COVID-19 to be a pandemic. COVID-19 is the disease caused by the novel beta coronavirus, SARS-CoV-2, an enveloped virus carrying a positive-sense, single-stranded RNA genome and a nucleocapsid with helical symmetry. The pathogenesis of COVID-19 is complex but begins with the binding of the spike (S) protein, shown in red in FIG. 1, to the Angiotensin Converting Enzyme 2 (ACE2) receptor on Type I and Type II pneumocytes in the lower respiratory tract. This binding event facilitates viral entry into respiratory epithelial cells, whereupon the viral genome replicates inside the cell and newly assembled virions bud from the plasma membrane to infect other cells.

Assumptions about downstream steps of COVID-19 pathogenesis derive from studies of the pathogenesis of the severe acute respiratory syndrome coronavirus, SARS-CoV, which also enters respiratory epithelium via ACE2 receptor and which caused an outbreak in 2002-2003. In addition to infecting respiratory epithelium, SARS-CoV can transiently infect macrophages and dendritic cells, which become activated by the infection². Damage to the lung tissue may be mediated by direct induction of cell death by the virus or by the innate and/or adaptive immune response to infection. At least three different lines of evidence suggest that a hyperactivated innate immune response is the primary mediator of the acute respiratory syndrome.

First, at least three different proteins encoded by SARS-CoV, the envelope (E) protein³, the open reading frame 3a (ORF3a) protein^{4,5}, and the ORF8b protein⁶, activate the NOD-, LRR- and pyrin domain-containing protein 3 (NLRP3) inflammasome, which stimulates inflammation through release of interleukin 1-beta and other cytokines from antigen-presenting cells. Interestingly, bats, the natural reservoir of the SARS coronaviruses, dampen NLRP3-mediated inflammation in response to both “sterile” insults or to multiple zoonotic virus infections and so do not get sick from these viruses despite persistent replication⁷⁻⁹. Second, patients who develop severe symptoms generally have higher levels of pro-inflammatory cytokines than do patients who recover from the disease with only mild symptoms¹⁰. Delayed type I interferon signaling promotes the accumulation of inflammatory monocyte/macrophages, resulting in elevated lung cytokine/chemokine levels, vascular leakage, and impaired virus-specific T cell responses¹¹. Third, worsening symptoms in week 2 after initial improvement occur concomitant with declining viral titers, suggesting that clinical deterioration is unrelated to uncontrolled viral replication but more related to immunopathological damage¹².

In contrast to the deleterious effects of innate immune system hyperactivation, there is evidence from animal models that an appropriate T cell response to SARS-CoV is both necessary and sufficient for viral clearance and recovery from infection¹. While either CD4+ and CD8+ T cells can accelerate virus clearance and enhance survival, virus-specific CD8+ T cells provide substantial protection from lethal infection by an attenuated strain of SARS-CoV (called MA15) in BALB/c mice². In addition to facilitating viral clearance, the adaptive immune response plays a major role in dampening innate immunity³. A reasonable hypothesis for the pathogenesis of severe or lethal COVID-19 is that lung damage and subsequent multi-organ failure results from excessive NLRP3-mediated innate immune activation and cytokine storm in the absence of a timely or effective response by virus-specific T cells, which would otherwise dampen the inflammation and clear the virus.

There is a significant unmet need for a treatment that would dampen the innate immune system hyperactivation and prevent further morbidity and mortality due to SARS-CoV-2 infection.

### SUMMARY OF THE INVENTION

There is some evidence that SARS from coronavirus results from inappropriate differentiation of virus-specific CD4⁺T cells into the type 2 (Tₕ2) subset, leading to inadequate activation of virus-specific, CD8⁺ cytotoxic T cells. Two pieces of evidence support this hypothesis. First, the attenuated MA15 strain of SARS-CoV is lethal in Tₕ2-biased BALB/c mice⁴ but is efficiently cleared by mice of the C57BL/6 or 129 strains^{5,6}, which are genetically biased toward type I, or Tₕ1, CD4+ T cell responses. Among the many possible differentiation states of CD4+ T cells, type 1, or Tₕ1, CD4+ T cells provide the optimal helper function for the differentiation and effector function of virus-specific, CD8+ cytotoxic T cells. Second, patients with severe acute respiratory syndrome from SARS-CoV exhibit a Tₕ2 predominance, marked by high serum concentrations of IL-10 and transforming growth factor-beta, and depletion of memory CD8+ T cells⁷.

Thus, administration of an agent that can push the immune response of an infected subject toward the type I, or Tₕ1, CD4+ T cell response, would lessen the immune hyperactivity and promote viral clearance.

In accordance with an embodiment, the present invention provides a method for post-exposure prophylaxis or treatment of a SARS coronaviral infection in a subject in need thereof comprising administering to the subject an effective amount of an agent or combination of agents that suppresses the NLRP3 inflammasome in the immune cells of the subject and promotes the differentiation of virus-specific CD4+ T cells into type 1 (Tₕ1) helper cells.

In accordance with an embodiment, the present invention provides a method for post-exposure prophylaxis or treatment of a SARS coronaviral infection in a subject in need thereof comprising administering to the subject an effective amount of an agent which inhibits tyrosine kinases, including Bruton’s tyrosine kinase and IL-2-inducible T cell kinase in the innate immune cells and CD4+ T cells, respectively, of the subject.

In accordance with an embodiment, the present invention provides a method for treatment of a SARS coronaviral infection in a subject in need thereof comprising administering to the subject an effective amount of a tyrosine kinase inhibitor to the subject.

In accordance with an embodiment, the present invention provides a method for post-exposure prophylaxis or treatment of a SARS coronaviral infection in a subject in need thereof comprising administering to the subject an effective amount of ibrutinib, acalabrutinib, zanubrutinib, or dasatinib to the subject.

In accordance with an embodiment, the present invention provides a method for treatment of a SARS coronaviral infection in a subject in need thereof comprising administering to the subject an effective amount of ibrutinib, acalabrutinib, zanubrutinib, or dasatinib and at least one or more other biologically active agents to the subject.

### BRIEF DESCRIPTION OF THE DRAWINGS

FIG. 1. Morphology of the coronavirus. The spike protein shown in red creates the appearance of a halo, or crown, on electron microscopy (from Wikipedia)

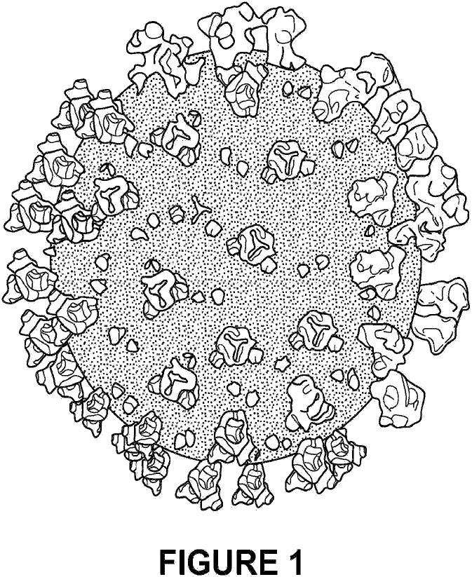

FIG. 2. Pathogenesis of lung injury in severe acute respiratory syndrome. From LB Ware et al.⁸

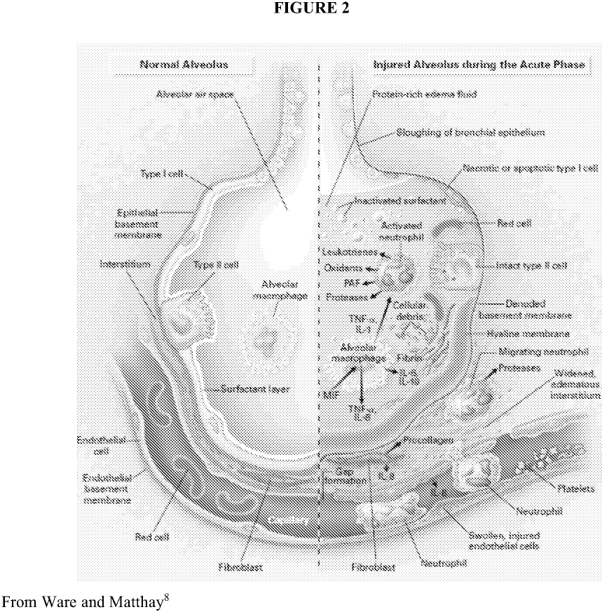

### DETAILED DESCRIPTION OF THE INVENTION

Without being limited to any particular theory, the present inventions are based on a central hypothesis that ibrutinib, a small molecule tyrosine kinase inhibitor that is approved to treat B cell malignancies, will improve the outcome of subjects exposed to or infected with SARS-CoV-2 by inhibiting virus-induced inflammation and by enhancing clearance of the virus.

Ibrutinib inhibits both the Bruton’s tyrosine kinase (BTK), and the IL-2 inducible T cell kinase (ITK). BTK is a key molecule in B cell development and signaling and inhibition of BTK by ibrutinib accounts for the drug’s activity in treating B cell malignancies. However, BTK physically interacts with components of the NLRP3 inflammasome⁹ and BTK inhibition by ibrutinib suppresses activation of the NLRP3 inflammasome^{9,10} and reduces IL-1 beta production by macrophages and neutrophils at sites of injury⁹. Intranasal administration of ibrutinib to influenza-infected mice attenuated lung inflammation, reduced the levels of inflammatory mediators TNF-alpha, IL-1 beta, and interleukin-6, and improved survival from virus-induced lung injury¹¹. ITK is required for differentiation of naive CD4+ T cells into Tₕ2 cells¹², and irreversible inhibition of ITK by ibrutinib subverts Tₕ2 differentiation and drives Tₕ1 immunity. Treatment of CLL patients with ibrutinib is accompanied by improvements in T cell number and function¹³ and the drug augments the activity of chimeric antigen-receptor modified T cells in killing their targets¹⁴.

It is believed that present inventive methods of administration of ibrutinib or other ITK inhibitors will enhance clearance of SARS-CoV-2 in subjects with COVID-19 by promoting the differentiation of virus-specific CD4+ T cells into Tₕ1 cells, enhancing the generation of virus-specific cytotoxic CD8+ T cells capable of eradicating the virus. In summary, we predict that ibrutinib will mitigate the severity of COVID-19 by inhibiting BTK-mediated activation of the NLRP3 inflammasome and consequent inflammation and lung injury and hasten recovery by enhancing virus-specific Tₕ1 differentiation and cytotoxic CD8+ T cell activity.

In accordance with an embodiment, the present invention provides a method for post-exposure prophylaxis or treatment of a SARS coronaviral infection in a subject in need thereof comprising administering to the subject an effective amount of an agent that suppresses the NLRP3 inflammasome in the immune cells of the subject.

As used herein, the term “NLRP3 inflammasome” means the NLRP3 inflammasome includes a sensor (NLRP3), an adaptor (ASC; also known as PYCARD) and an effector (caspase 1). NLRP3 is a tripartite protein that contains an amino-terminal pyrin domain (PYD), a central NACHT domain (domain present in NAIP, CIITA, HET-E and TP1) and a carboxy-terminal leucine-rich repeat domain (LRR domain). The NACHT domain has ATPase activity that is vital for NLRP3 self-association and function⁸, whereas the LRR domain is thought to induce autoinhibition by folding back onto the NACHT domain. ASC has two protein interaction domains, an amino-terminal PYD and a carboxy-terminal caspase recruitment domain (CARD). Full-length caspase 1 has an amino-terminal CARD, a central large catalytic domain (p20) and a carboxy-terminal small catalytic subunit domain (p10). Upon stimulation, NLRP3 oligomerizes through homotypic interactions between NACHT domains. Oligomerized NLRP3 recruits ASC through homotypic PYD-PYD interactions and nucleates helical ASC filament formation, which also occurs through PYD-PYD interactions. Multiple ASC filaments coalesce into a single macromolecular focus, known as an ASC speck. Assembled ASC recruits caspase 1 through CARD-CARD interactions and enables proximity-induced caspase 1 self-cleavage and activation. Caspase 1 clustered on ASC selfcleaves at the linker between p20 and p10 to generate a complex of p33 (comprising the CARD and p20) and p10, which remains bound to ASC and is proteolytically active. Further processing between the CARD and p20 releases p20-p10 from ASC. The released p20-p10 heterotetramer is unstable in cells, hence terminating its protease activity. Recently, NIMArelated kinase 7 (NEK7), a serine-threonine kinase that is known to be involved in mitosis, was found to be essential for NLRP3 inflammasome activation. NEK7 specifically interacts with NLRP3, but not the other inflammasome sensors NLRC4 (NOD-, LRR- and CARDcontaining 4) or interferon-inducible protein AIM2. Upon inflammasome activation, the NEK7-NLRP3 interaction increases, and NEK7 oligomerizes with NLRP3 into a complex that is essential for ASC speck formation and caspase 1 activation. Thus, NEK7 appears to be a core component specific to the NLRP3 inflammasome.

As used herein, the term “agent that suppresses the NLRP3 inflammasome” means any biologically active agent that inhibits or otherwise mitigates activation of the NLRP3 inflammasome and consequent local or systemic inflammation, potential lung damage or dysfunction, systemic inflammatory response syndrome, or multi-organ dysfunction.

In accordance with an embodiment, the present invention provides an ITK inhibitor or a salt, solvate, stereoisomer, or derivative thereof, and a pharmaceutically acceptable carrier, for use as a medicament, preferably for use as a NLRP3 inflammasome inhibitor in a mammalian cell or population of cells, more preferably for use as a treatment in a subject suffering from a viral infection, preferably a coronavirus infection.

As used herein, the term “SARS coronavirus infection” means a subject who is infected with one or more coronaviruses (CoVs). CoVs are enveloped positive-sense RNA viruses, are characterized by club-like spikes that project from their surface, an unusually large RNA genome, and a unique replication strategy. Coronaviruses cause a variety of diseases in mammals and birds ranging from enteritis in cows and pigs and upper respiratory disease chickens to potentially lethal human respiratory infections.

The initial attachment of the CoV virion to the host cell is initiated by interactions between the S protein and its receptor. The sites of receptor binding domains (RBD) within the S1 region of a coronavirus S protein vary depending on the virus, with some having the RBD at the N-terminus of S1 (MHV) while others (SARS-CoV) have the RBD at the C-terminus of S1. The S-protein/receptor interaction is the primary determinant for a coronavirus to infect a host species and also governs the tissue tropism of the virus. Many coronaviruses utilize peptidases as their cellular receptor. It is unclear why peptidases are used, as entry occurs even in the absence of the enzymatic domain of these proteins. Many α-coronaviruses utilize aminopeptidase N (APN) as their receptor, SARS-CoV, SARS-CoV-2, and HCoV-NL63 use angiotensin-converting enzyme 2 (ACE2) as their receptor, MHV enters through CEACAM1, and the recently identified MERS-CoV binds to dipeptidyl-peptidase 4 (DPP4) to gain entry into human cells.

In some embodiments, the subject is infected with SARS-CoV-2, which causes the COVID-19 disease.

In some embodiments, the agent that suppresses the NLRP3 inflammasome is an agent which inhibits tyrosine kinase. In certain embodiments, the agent suppresses Bruton’s tyrosine kinase. In other embodiments, the agent suppresses IL-2-inducible T cell kinases.

In accordance with another embodiment, the present invention provides a method for post-exposure prophylaxis or treatment of a SARS coronaviral infection in a subject in need thereof comprising administering to the subject an effective amount of an agent which suppresses Bruton’s tyrosine kinase and/or IL-2-inducible T cell kinase in the immune cells of the subject.

In accordance with an embodiment, the present invention provides a method for post-exposure prophylaxis or treatment of a SARS coronaviral infection in a subject in need thereof comprising administering to the subject an effective amount of a tyrosine kinase inhibitor to the subject.

As used herein, the term “tyrosine kinase inhibitor” means a family of small molecules or peptides with the ability to inhibit either cytosolic or receptor tyrosine kinases. Inhibition by this class of agents is through direct competition for ATP binding to the tyrosine kinase (genistein, lavendustin C, PP1-AG1872, PP2-AG1879, SU6656, CGP77675, PD166285, imatinib, erlotinib, gefitinib), allosteric inhibition of the tyrosine kinase (lavendustin A), inhibition of ligand binding to receptor tyrosine kinases (e.g., cetuximab), inhibition of tyrosine kinase interaction with other proteins (e.g., UCS15A, p60-v-Src inhibitor peptide) or destabilization of the tyrosine kinase (e.g., herbimycin A and radicicol).

TABLE 1

Approved Tyrosine Kinase Inhibitors

| TKI | Time to market | Development company | Target | Application of disease |
|---|---|---|---|---|
| Imatinib | 2001 | Novartis | Abl, PDGFR, SCFR | CML, GIST |
| Gefitinib | 2003 | AstraZeneca | EGFR | NSCLC |
| Nilotinib | 2004 | Novartis | Bcr-Abl, PDGFR | CML |
| Sorafenib | 2005 | Bayer | Raf, VEGFR, PDGER | Advanced RCC |
| Sunitinib | 2006 | Pfizer | PDGFR, VEGFR, | RCC, Advanced |
| Dasatinib | 2006 | Bristol-Myers Squibb | Bcr-Abl, SRC, PDGFR | CML |
| Lapatinib | 2007 | GlaxoSmithKline | EGFR | Breast cancer |
| Pazopanib | 2009 | GlaxoSmithKline | VEGFR, PDGFR, FGFR | Advanced RCC,STS,NSCL C |
| Crizotinib | 2011 | Pfizer | ALK | NSCLC |
| Ruxolitinib | 2011 | Novartis | JAK1, JAK2 | myelofibrosis |
| vandetanib | 2011 | AstraZeneca | VEGFR, EGFR | Advanced Thyroid cancer |
| Axitinib | 2012 | Pfizer | VEGFR | Advanced RCC |
| Bosutinib | 2012 | Wyeth | Abl, SRC | CML |
| Afatinib | 2013 | Boehringer Ingelheim | EGFR | NSCLC |
| Erlotinib | 2013 | Roche | EGFR | NSCLC |
| Ceritinib | 2014 | Novartis | ALK | NSCLC |
| Osimertinib | 2015 | AstraZeneca | EGFR | NSCLC |
| Lenvatinib | 2015 | Eisai | VEGFR | DTC |
| Alectinib | 2015 | Roche | ALK | NSCLC |
| Ibrutinib | 2016 | Abbvie | BTK | MCL, CLL |
| Regorafenib | 2017 | Bayer | VEGFR, EGFR | HCC, CRC,GIST |
| Neratinib | 2017 | Puma | HER2 | Breast cancer |
| Brigatinib | 2017 | Ariad | ALK | NSCLC |
| Acalabrutinib | 2018 (?) | Astra Zeneca | BTK | CLL, SLL |
| Zanubrutinib | 2019 | Beigene | BTK | MCL |

In accordance with an embodiment, the present invention provides a method for post-exposure prophylaxis or treatment of a SARS coronaviral infection in a subject in need thereof comprising administering to the subject an effective amount of ibrutinib, acalabrutinib, zanubrutinib, or dasatinib to the subject.

As used herein, the term “ibrutinib” means is an orally bioavailable, smallmolecule inhibitor of Bruton’s tyrosine kinase (BTK) with potential antineoplastic activity. Upon oral administration, ibrutinib binds to and irreversibly inhibits BTK activity, thereby preventing both B-cell activation and B-cell-mediated signaling. This leads to an inhibition of the growth of malignant B cells that overexpress BTK. BTK, a member of the src-related BTK/Tec family of cytoplasmic tyrosine kinases, is required for B cell receptor signaling, plays a key role in B-cell maturation, and is overexpressed in a number of B-cell malignancies. The compound ibrutinib has the following structure:

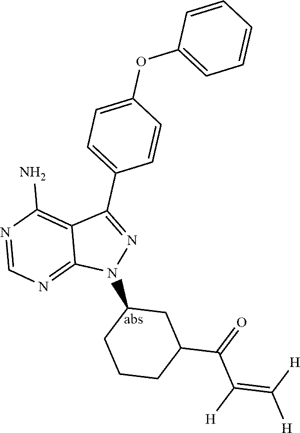

It will be understood that the term “ibrutinib” includes all salts, solvates, and stereoisomers of the compound of formula I.

The compound acalabrutinib has the following structure:

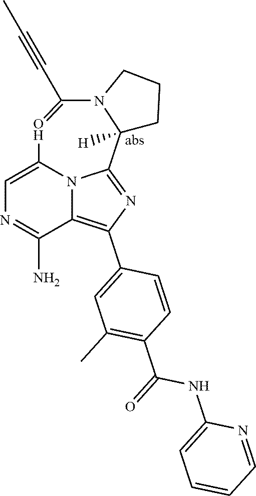

It will be understood that the term “acalabrutinib” includes all salts, solvates, and stereoisomers of the compound of formula II.

The compound zanubrutinib has the following structure:

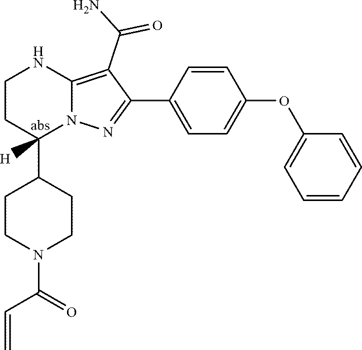

It will be understood that the term “zanubrutinib” includes all salts, solvates, and stereoisomers of the compound of formula III.

The compound dasatinib has the following structure:

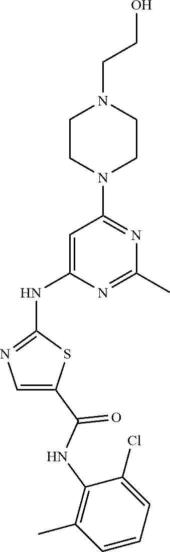 [SMILES: Cc1nc(Nc2ncc(C(=O)Nc3c(C)cccc3Cl)s2)cc(N2CCN(CCO)CC2)n1]

It will be understood that the term “zanubrutinib” includes all salts, solvates, and stereoisomers of the compound of formula IV.

Accordingly, included within the compounds and derivatives of the present invention are the tautomeric forms of the disclosed compounds, isomeric forms including enantiomers, stereoisomers, and diastereoisomers, and the pharmaceutically-acceptable salts thereof. The term “pharmaceutically acceptable salts” embraces salts commonly used to form alkali metal salts and to form addition salts of free acids or free bases, such as those used to improve water solubility. Examples of acids, which may be employed to form pharmaceutically acceptable acid addition salts, include such inorganic acids as hydrochloric acid, sulphuric acid and phosphoric acid, and such organic acids as maleic acid, succinic acid and citric acid. Other pharmaceutically acceptable salts include salts with alkali metals or alkaline earth metals, such as sodium, potassium, calcium and magnesium, or with organic bases, such as dicyclohexylamine. Suitable pharmaceutically acceptable salts of the compounds of the present invention include, for example, acid addition salts which may, for example, be formed by mixing a solution of the compound according to the invention with a solution of a pharmaceutically acceptable acid, such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. All of these salts may be prepared by conventional means by reacting, for example, the appropriate acid or base with the corresponding compounds of the present invention.

Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicines, the salts of the compounds of the present invention should be pharmaceutically acceptable salts. Other salts may, however, be useful in the preparation of the compounds according to the invention or of their pharmaceutically acceptable salts.

In addition, embodiments of the invention include hydrates of the compounds of the present invention. The term “hydrate” includes but is not limited to hemihydrate, monohydrate, dihydrate, trihydrate and the like. Hydrates of the compounds of the present invention may be prepared by contacting the compounds with water under suitable conditions to produce the hydrate of choice.

As used herein, the term “treat,” as well as words stemming therefrom, includes preventative as well as disorder remitative treatment. The terms “reduce,” “suppress,” “prevent,” and “inhibit,” as well as words stemming therefrom, have their commonly understood meaning of lessening or decreasing. These words do not necessarily imply 100% or complete treatment, reduction, suppression, or inhibition.

It will be understood by those of ordinary skill in the art that treatment in the present inventive methods, means to reduce the inflammation in the lungs of the subject infected with a SARS coronavirus, such as SARS-CoV2 which causes COVID-19 disease. Examples of such treatment would include a subject having: 1) decreased oxygen dependence (administered oxygen) or progressing from oxygen dependence to oxygen independence, reduced deterioration or improved pulmonary function as measured by tests such as the diffusing capacity of carbon monoxide; 2) reduced number or extent of infiltrates, or delayed progression of infiltrates, on chest imaging; 3) improvement or reduced deterioration of the following clinical tests: red blood cell count, platelet count, white blood cell count, absolute neutrophil count, absolute lymphocyte count, serum albumin, alanine aminotransferase, creatinine, lactate dehydrogenase, creatine kinase, high-sensitivity cardiac troponin, prothrombin time, d-dimer, serum ferritin, procalcitonin, interleukin 1-beta, interleukin-6; or 4) improvement or reduced deterioration of the following clinical parameters: fever, cough, respiratory rate, pulse, or Sequential Organ Failure Assessment (SOFA) score¹⁵.

Other examples of treatment outcomes include, but are not limited to, enhance clearance of SARS-CoV-2 in patients with COVID-19 by promoting the differentiation of virus-specific CD4+ T cells into Tₕ1 cells, enhancing the generation of virus-specific cytotoxic CD8+ T cells capable of eradicating the virus, and increasing lung function.

With respect to pharmaceutical compositions described herein for use in the present invention, the pharmaceutically acceptable carrier can be any of those conventionally used, and is limited only by physico-chemical considerations, such as solubility and lack of reactivity with the active compound(s), and by the route of administration. The pharmaceutically acceptable carriers described herein, for example, vehicles, adjuvants, excipients, and diluents, are well known to those skilled in the art and are readily available to the public. Examples of the pharmaceutically acceptable carriers include soluble carriers such as known buffers which can be physiologically acceptable (e.g., phosphate buffer) as well as solid compositions such as solid-state carriers or latex beads. It is preferred that the pharmaceutically acceptable carrier be one which is chemically inert to the active agent(s), and one which has little or no detrimental side effects or toxicity under the conditions of use.

The carriers or diluents used herein may be solid carriers or diluents for solid formulations, liquid carriers or diluents for liquid formulations, or mixtures thereof.

Solid carriers or diluents include, but are not limited to, gums, starches (e.g., corn starch, pregelatinized starch), sugars (e.g., lactose, mannitol, sucrose, dextrose), cellulosic materials (e.g., microcrystalline cellulose), acrylates (e.g., polymethylacrylate), calcium carbonate, magnesium oxide, talc, or mixtures thereof.

For liquid formulations, pharmaceutically acceptable carriers may be, for example, aqueous or non-aqueous solutions, suspensions, emulsions or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Parenteral vehicles (for subcutaneous, intravenous, intraarterial, or intramuscular injection) include, for example, sodium chloride solution, Ringer’s dextrose, dextrose and sodium chloride, lactated Ringer’s and fixed oils. Formulations suitable for parenteral administration include, for example, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Intravenous vehicles include, for example, fluid and nutrient replenishers, electrolyte replenishers such as those based on Ringer’s dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions

In addition, in an embodiment, the compounds used in the methods of the present invention may further comprise, for example, binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmelose sodium, crospovidone, guar gum, sodium starch glycolate), buffers (e.g., Tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., cremophor, glycerol, polyethylene glycerol, benzalkonium chloride, benzyl benzoate, cyclodextrins, sorbitan esters, stearic acids), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweetners (e.g., aspartame, citric acid), preservatives (e.g., thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates), and/or adjuvants.

The choice of carrier will be determined, in part, by the particular compound, as well as by the particular method used to administer the compound. Accordingly, there are a variety of suitable formulations of the pharmaceutical composition of the invention. The following formulations for parenteral, subcutaneous, intravenous, intramuscular, intraarterial, intrathecal and interperitoneal administration are exemplary, and are in no way limiting. More than one route can be used to administer the compounds, and in certain instances, a particular route can provide a more immediate and more effective response than another route.

Suitable soaps for use in parenteral formulations include, for example, fatty alkali metal, ammonium, and triethanolamine salts, and suitable detergents include, for example, (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain from about 0.5% to about 25% by weight of the compounds in solution. Preservatives and buffers may be used. In order to minimize or eliminate irritation at the site of injection, such compositions may contain one or more nonionic surfactants, for example, having a hydrophile-lipophile balance (HLB) of from about 12 to about 17. The quantity of surfactant in such formulations will typically range from about 5% to about 15% by weight. Suitable surfactants include, for example, polyethylene glycol sorbitan fatty acid esters, such as sorbitan monooleate and the high molecular weight adducts of ethylene oxide with a hydrophobic base, formed by the condensation of propylene oxide with propylene glycol.

The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Injectable formulations are in accordance with the invention. The requirements for effective pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Trissel, 15th ed., pages 622-630 (2009)).

In an embodiment, the term “administering” means that the compounds of the present invention are introduced into a subject, preferably a subject receiving treatment for a a coronalviral disease, and the compounds are allowed to come in contact with the one or more disease related cells or population of cells in vivo.

As defined herein, in another embodiment, the term “contacting” means that the one or more compounds of the present invention are introduced into a sample having at least one virus infected cell and appropriate enzymes or reagents, in a test tube, flask, tissue culture, chip, array, plate, microplate, capillary, or the like, and incubated at a temperature and time sufficient to permit binding and uptake of the at least one compound to the virus infected cell. Methods for contacting the samples with the compounds, and other specific binding components are known to those skilled in the art, and may be selected depending on the type of assay protocol to be run. Incubation methods are also standard and are known to those skilled in the art.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

In accordance with an embodiment, the present invention provides a method for treatment of a SARS coronaviral infection in a subject in need thereof comprising administering to the subject an effective amount of ibrutinib, acalabrutinib, zanubrutinib, or dasatinib and at least one or more other biologically active agents to the subject.

The biologically active agent may vary widely with the intended purpose for the composition. The term active is art-recognized and refers to any moiety that is a biologically, physiologically, or pharmacologically active substance that acts locally or systemically in a subject. Examples of biologically active agents, that may be referred to as “drugs”, are described in well-known literature references such as the Merck Index, the Physicians’ Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a biologically active agent may be used which are capable of being released the subject composition, for example, into adjacent tissues or fluids upon administration to a subject.

Further examples of biologically active agents include, without limitation, enzymes, receptor antagonists or agonists, hormones, growth factors, autogenous bone marrow, antibiotics, antimicrobial agents, and antibodies.

Non-limiting examples of biologically active agents include following: adrenergic blocking agents, anabolic agents, androgenic steroids, antacids, anti-asthmatic agents, antiallergenic materials, anti-cholesterolemic and anti-lipid agents, anti-cholinergics and sympathomimetics, anti-coagulants, anti-convulsants, anti-diarrheal, anti-emetics, antihypertensive agents, anti-infective agents, anti-inflammatory agents such as steroids, nonsteroidal anti-inflammatory agents, anti-malarials, anti-manic agents, anti-nauseants, antineoplastic agents, anti-obesity agents, anti-parkinsonian agents, anti-pyretic and analgesic agents, anti-spasmodic agents, anti-thrombotic agents, anti-uricemic agents, anti-anginal agents, antihistamines, anti-tussives, appetite suppressants, benzophenanthridine alkaloids, biologicals, cardioactive agents, cerebral dilators, coronary dilators, decongestants, diuretics, diagnostic agents, erythropoietic agents, estrogens, expectorants, gastrointestinal sedatives, agents, hyperglycemic agents, hypnotics, hypoglycemic agents, ion exchange resins, laxatives, mineral supplements, mitotics, mucolytic agents, growth factors, neuromuscular drugs, nutritional substances, peripheral vasodilators, progestational agents, prostaglandins, psychic energizers, psychotropics, sedatives, stimulants, thyroid and anti-thyroid agents, tranquilizers, uterine relaxants, vitamins, antigenic materials, and prodrugs.

Various forms of the biologically active agents may be used. These include, without limitation, such forms as uncharged molecules, molecular complexes, salts, ethers, esters, amides, prodrug forms and the like, which are biologically activated when implanted, injected or otherwise placed into a subject.

In certain embodiments, other materials may be incorporated into subject compositions in addition to one or more biologically active agents. For example, plasticizers and stabilizing agents known in the art may be incorporated in compositions of the present invention.

Typically, an attending physician will decide the dosage of the compositions described herein with which to treat each individual subject, taking into consideration a variety of factors, such as age, body weight, general health, diet, sex, compound to be administered, route of administration, and the severity of the condition being treated. By way of example, and not intending to limit the invention, the dose of the compositions of the present invention can be about 0.001 to 1000 mg/kg body weight of the subject being treated, from about 0.01 to 100 mg/kg body weight, from about 0.1 mg/kg to 10 mg/kg, and from about 0.5 mg to 5 mg/kg body weight. In another embodiment, the dose of the compositions of the present invention can be at a concentration from about 1 nM to 100 mM, preferably from about 10 µM to 50 mM, more preferably from about 100 µM to 5 mM.

In some embodiments, the dosage of ibrutinib administered to the subject is between about 100 mg/day to 1000 mg/day for 1 to 2 or more weeks. In some embodiments, the amount of ibrutinib administered to the subject is between about 400-500 mg/day.

As used herein, the term “effective amount” is an equivalent phrase refers to the amount of a therapy (e.g., a prophylactic or therapeutic agent), which is sufficient to reduce the severity and/or duration of a disease, ameliorate one or more symptoms thereof, prevent the advancement of a disease or cause regression of a disease, or which is sufficient to result in the prevention of the development, recurrence, onset, or progression of a disease or one or more symptoms thereof, or enhance or improve the prophylactic and/or therapeutic effect(s) of another therapy (e.g., another therapeutic agent) useful for treating a disease. For example, a treatment of interest can suppress the NLRP3 inflammasome, based on decreased inflammation in the lungs of a subject having a SARS coronaviral infection, by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In another embodiment, an effective amount of a therapeutic or a prophylactic agent of interest reduces the symptoms of a disease, such as inhibition of BTK in the lungs of a subject having a SARS coronaviral infection by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. Also used herein as an equivalent is the term, “therapeutically effective amount.

In another embodiment, an effective amount of a therapeutic or a prophylactic agent of interest hastens elimination of the viral infection by inhibiting ITK in T lymphocytes by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.”

An article of manufacture containing materials useful for the treatment of the disorders described above is provided. The article of manufacture comprises a container and a label. Suitable containers include, for example, bottles, vials, syringes and test tubes. The containers may be formed from a variety of materials such as glass or plastic. The container holds a composition which is effective for preventing or treating, for example, a wound or a joint disease and may have a sterile access port (for example, the container may be a vial having a stopper pierceable by a hypodermic injection needle). The label on or associated with the container indicates that the composition is used for treating the condition of choice. The article of manufacture may further comprise a second container comprising a pharmaceutically acceptable buffer, such as phosphate-buffered saline, Ringer’s solution and dextrose solution. It may further include other materials desirable from a commercial and user standpoint, including buffers, diluents, filters, needles, syringes and package inserts with instructions for use.

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### EXAMPLES

A prospective, randomized trial, document the percentage of patients experiencing pulmonary deterioration from COVID-19.

Eligibility criteria:
Age ≥60
Documented COVID-19
   a. Fever
   b. Positive nasopharyngeal swab test for SARS-CoV-2
Resting O₂ saturation ≥93%

### Treatment Plan

Patients are randomized to best supportive care versus ibrutinib 420 mg/day x 2 weeks, beginning the day of positive nasopharyngeal swab. O₂ saturation will be monitored at least daily during the two-week period.

Positive outcomes will be lower percentage of treated subjects progressing to oxygen dependence compared with subjects receiving supportive care. Shorter duration of symptoms for the treated subjects. Less pulmonary symptoms during treatment comparted to subjects receiving supportive care.

Primary endpoint: Percentage of patients progressing from oxygen independence (resting O₂ saturation ≥93%) to oxygen dependent (resting O₂ saturation <90%).All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### References

1. Ware LB, Matthay MA. The acute respiratory distress syndrome. The New England journal of medicine 2000;342:1334-49.

2. Spiegel M, Schneider K, Weber F, Weidmann M, Hufert FT. Interaction of severe acute respiratory syndrome-associated coronavirus with dendritic cells. The Journal of general virology 2006;87:1953-60.

3. Nieto-Torres JL, Verdiá-Báguena C, Jimenez-Guardeño JM, et al. Severe acute respiratory syndrome coronavirus E protein transports calcium ions and activates the NLRP3 inflammasome. Virology 2015;485:330-9.

4. Siu K-L, Yuen K-S, Castaño-Rodriguez C, et al. Severe acute respiratory syndrome coronavirus ORF3a protein activates the NLRP3 inflammasome by promoting TRAF3-dependent ubiquitination of ASC. FASEB journal : official publication of the Federation of American Societies for Experimental Biology 2019;33:8865-77.

5. Chen IY, Moriyama M, Chang MF, Ichinohe T. Severe acute respiratory syndrome coronavirus viroporin 3a activates the NLRP3 inflammasome. Frontiers in Microbiology 2019;10.

6. Shi C-S, Nabar NR, Huang N-N, Kehrl JH. SARS-Coronavirus Open Reading Frame-8b triggers intracellular stress pathways and activates NLRP3 inflammasomes. Cell Death Discov 2019;5:101-.

7. Xie J, Li Y, Shen X, et al. Dampened STING-Dependent Interferon Activation in Bats. Cell host & microbe 2018;23:297-301.e4.

8. Hayman DTS. Bat tolerance to viral infections. Nature Microbiology 2019;4:728-9.

9. Ahn M, Anderson DE, Zhang Q, et al. Dampened NLRP3-mediated inflammation in bats and implications for a special viral reservoir host. Nature Microbiology 2019;4:789-99.

10. Channappanavar R, Perlman S. Pathogenic human coronavirus infections: causes and consequences of cytokine storm and immunopathology. Seminars in immunopathology 2017;39:529-39.

11. Channappanavar R, Fehr AR, Vijay R, et al. Dysregulated Type I Interferon and Inflammatory Monocyte-Macrophage Responses Cause Lethal Pneumonia in SARS-CoV-Infected Mice. Cell Host and Microbe 2016;19:181-93.

12. Peiris JSM, Chu CM, Cheng VCC, et al. Clinical progression and viral load in a community outbreak of coronavirus-associated SARS pneumonia: a prospective study. Lancet (London, England) 2003;361:1767-72.

13. Zhao J, Zhao J, Perlman S. T cell responses are required for protection from clinical disease and for virus clearance in severe acute respiratory syndrome coronavirus-infected mice. Journal of virology 2010;84:9318-25.

14. Channappanavar R, Fett C, Zhao J, Meyerholz DK, Perlman S. Virus-specific memory CD8 T cells provide substantial protection from lethal severe acute respiratory syndrome coronavirus infection. Journal of Virology 2014;88:11034-44.

15. Kim KD, Zhao J, Auh S, et al. Adaptive immune cells temper initial innate responses. Nature medicine 2007;13:1248-52.

16. Roberts A, Deming D, Paddock CD, et al. A mouse-adapted SARS-coronavirus causes disease and mortality in BALB/c mice. PLoS pathogens 2007;3:e5-e.

17. Zhao J, Zhao J, Legge K, Perlman S. Age-related increases in PGD(2) expression impair respiratory DC migration, resulting in diminished T cell responses upon respiratory virus infection in mice. The Journal of clinical investigation 2011;121:4921-30.

18. Frieman MB, Chen J, Morrison TE, et al. SARS-CoV pathogenesis is regulated by a STAT1 dependent but a type I, II and III interferon receptor independent mechanism. PLoS pathogens 2010;6:e1000849-e.

19. Huang J-L, Huang J, Duan Z-H, et al. Th2 predominance and CD8+ memory T cell depletion in patients with severe acute respiratory syndrome. Microbes and infection 2005;7:427-36.

20. Ito M, Shichita T, Okada M, et al. Bruton’s tyrosine kinase is essential for NLRP3 inflammasome activation and contributes to ischaemic brain injury. Nature communications 2015;6:7360-.

21. Liu X, Pichulik T, Wolz O-O, et al. Human NACHT, LRR, and PYD domain-containing protein 3 (NLRP3) inflammasome activity is regulated by and potentially targetable through Bruton tyrosine kinase. J Allergy Clin Immunol 2017;140:1054-67.e10.

22. Dubovsky JA, Beckwith KA, Natarajan G, et al. Ibrutinib is an irreversible molecular inhibitor of ITK driving a Th1-selective pressure in T lymphocytes. Blood 2013;122:2539-49.

23. Long M, Beckwith K, Do P, et al. Ibrutinib treatment improves T cell number and function in CLL patients. J Clin Invest 2017;127:3052-64.

24. Fraietta JA, Beckwith KA, Patel PR, et al. Ibrutinib enhances chimeric antigen receptor T-cell engraftment and efficacy in leukemia. Blood 2016;127:1117-27.

1. A method for treatment of a SARS coronavirus infection in a subject in need thereof comprising administering to the subject an effective amount of an agent that suppresses the NLRP3 inflammasome in the immune cells of the subject, with or without an agent that inhibits interleukin-2 inducible T cell kinase (ITK) in the T cells of the subject, thereby treating the SARS infection.

2. A method for treatment of a SARS coronavirus infection in a subject in need thereof comprising administering to the subject an effective amount of an agent which suppresses Bruton’s tyrosine kinase in the immune cells of the subject, with or without an agent that inhibits interleukin-2 inducible T cell kinase in the T cells of the subject, thereby treating the SARS infection.

3. A method for treatment of a SARS coronavirus infection in a subject in need thereof comprising administering to the subject an effective amount of a tyrosine kinase inhibitor to the subject, thereby treating the SARS infection.

4. The method of claim 1, wherein the SARS coronavirus infection is due to infection with SARS-CoV-2.

5. The method of claim 1, wherein the tyrosine kinase inhibitor is ibrutinib.

6. The method of claim 1, wherein the Bruton’s tyrosine kinase inhibitor is acalabrutinib or zanubrutinib, and the ITK inhibitor is BMS-509744 or GNE-4997.

7. The method of claim 5, wherein the ibrutinib is administered to the subject at the onset of infection.

8. The method of claim 7, wherein the dosage of ibrutinib administered to the subject is between about 100 mg/day to 1000 mg/day.

9. The method of claim 8, wherein the dosage is about 420 mg/day.

10. The method of claim 7, wherein the ibrutinib is administered to the subject for about 10 to 20 days.

11. The method of claim 9, wherein ibrutinib is administered to the subject for about 14 days.
